(19) United States
(12) Patent Application Publication
(10) Pub. No.: US 2008/0108545 A1
(43) Pub. Date: May 8, 2008

(54) Peptide Nucleic Acid Conjugates and Uses Thereof

(75) Inventors: Roger Michael Eccles, Mosgiel (NZ); Aleksandra Filipovska, Wangara (Western Australia) (AU); Robin A.J. Smith, Dunedin (NZ); Michael Patrick Murphy, Cambridge (GB)

Correspondence Address: JACOBSON HOLMAN PLLC, 400 SEVENTH STREET N.W., SUITE 600, WASHINGTON, DC, 20004, US

(73) Assignee: UNIVERSITY OF OTAGO, Dunedin (NZ)

(21) Appl. No.: 10/575,780

(22) PCT Filed: Oct. 15, 2004
(86) PCT No.: PCT/NZ04/00255

§ 371 Date: Sep. 25, 2007

(30) Foreign Application Priority Data
Oct. 17, 2003 (NZ) 528966

(51) Int. Cl. A61K 31/70; C07K 14/00; C07K 1/00; A61P 7/00; A61P 1/00; A61K 38/00

(52) U.S. Cl. 514/2; 530/395; 530/402; 514/44

(57) ABSTRACT

The invention provides a triphenylphosphonium peptide nucleic acid conjugate. The conjugate comprises a peptide nucleic acid linked via a disulfide bond, to a triphenylphosphonium group. Conjugates of the invention can be used to deliver PNA oligomers across the plasma membrane into cells.

### TECHNICAL FIELD

The invention relates to triphenylphosphonium (TPP)-peptide nucleic acid (PNA) conjugates and uses thereof.

### BACKGROUND OF THE INVENTION

Peptide nucleic acid oligomers (PNAs) are structural analogues of oligonucleotides that can mimic DNA and RNA. PNAs comprise a pseudo-peptide backbone to which nucleobases are attached. A commonly used PNA replaces the deoxyribose-phosphate linkages in DNA with an uncharged polyamide backbone comprised of N-(2-aminoethyl) glycine units (Egholm et al., 1993). As this modification to the backbone does not alter the spacing of the bases related to DNA and RNA, PNAs can be designed to be complementary to a particular mRNA transcript permitting the antisense oligomer to undergo Watson-Crick hybridisation with its target (Egholm et al., 1993). This results in mRNA inactivation through steric blocking of the spliceosome or ribosome complex and consequently, specific inhibition of the synthesis of a particular protein product.

Unlike other antisense agents, PNAs can be used as antigene agents to target the DNA sequence of a gene through the formation of a triple-helix. This is potentially a more direct way to inhibit gene expression with fewer potential cellular targets than amplifiable mRNAs. Alternatively, as antisense agents, PNAs bind mRNAs more efficiently than any other available antisense agent (Knudsen et al., 1996).

As such, PNAs can be used as tools to manipulate gene expression and may have application as therapies for a range of diseases, however, a disadvantage of the application of PNAs as antisense agents is their low rate of membrane permeation (Eriksson et al., 1996).

Several attempts have been made to circumvent this difficulty. For example, PNAs have been conjugated to cell penetrating peptides such as penetratin, Tat and transportan for delivery to the cytoplasm or nucleus of cells (Simmons et al., 1997; Eriksson et al., 2001, WO 00/05302). However, the synthesis of these peptides is expensive and complex.

PNAs have also been encapsulated in cationic liposomes to improve cellular uptake (Ljungstrom et al., 1999). However, this method is very dependent on the cell type and also dependent on the PNA sequence, and cellular uptake is quite slow. In addition, liposomes themselves induce a stress-response in cells and are cytotoxic at high concentrations.

Therefore, there is still the need for a simple and effective way to deliver PNA oligomers across the plasma membrane to the cytoplasm of the cell.

It has been shown that PNAs conjugated to a lipophilic cation easily pass through lipid bilayers. For example, the phosphonium cation catalyses the uptake of PNA through lipid bilayers as even the relatively small membrane potential of the plasma membrane 30-60 mV (negative inside) helps deliver cargo conjugated to lipophilic cations into the cytoplasm. However, the large membrane potential (−150 to −170 mV) across the mitochondrial membrane causes lipophilic cation-PNA conjugates to selectively localise to mitochondria within cells (Muratovska et. al., 2001; WO 99/26954). Consequently, techniques utilising known lipophilic cation-PNA conjugates are limited to the selective manipulation of mitochondrial DNA.

Accordingly, it is an object of the present invention to provide lipophilic cation conjugates useful for transporting PNA oligomers into cells without being taken into the mitochondria and/or at least to provide the public with a useful choice.

### SUMMARY OF THE INVENTION

In one aspect, the invention provides a conjugate of formula I

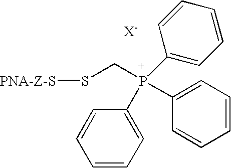

wherein L is a linker group, S-Z is a thiol-containing attachment group, X⁻ is an optional anion, and PNA is a peptide nucleic acid.

Preferably, the linker group is (C₁-C₃₀) alkylene or substituted (C₁-C₃₀) alkylene. More preferably the linker group is (C₂-C₂₀) alkylene or substituted (C₂-C₂₀) alkylene. Even more preferably, the linker group is (C₂-C₁₀) alkylene or substitute (C₂-C₁₀) alkylene. Most preferably, the linker group is (C₃-C₅) butylene or substituted (C₃-C₅) butylene.

Preferably, Z is selected so that S-Z is a cysteinyl, homocysteinyl or an aminothiol compound attached to a suitable linking group for linking to the PNA residue.

More preferably, Z is selected so that S-Z is a cysteinyl, homocysteinyl or an aminothiol compound attached to an 8-amino-3,6-dioxanoic acid residue.

The phenyl groups of the triphenylphosphonium moiety may be optionally substituted with alkyl groups or any other group provided that the conjugate remains hydrophobic enough to transfer across the cell membrane.

The anion X⁻ is optionally present as required for overall electrical neutrality.

Preferably, the anion is an inorganic anion derived from hydrochloric, hydrobromic, hydroiodic, sulfuric, sulfurous, nitric, nitrous, phosphoric or phosphorous acid, or from an alkylsulfonic or an arylsulfonic acid.

More preferably, the anion is a halo anion, most preferably bromide.

Preferably, the PNA is attached to a molecular tag, atom or reporter molecule such as an affinity label (for example, biotin (bio), streptavidin and the like) through a spacer group such as one or more 8-amino-3,6-dioxanoic acid residues.

More preferably the reporter is a fluorophore (such as for example Cy3, Cy5 and Cy2), most preferably, fluroscein (flu).

Preferably, the PNA contains between about 3 to 25 nucleotides, more preferably between about 5 to 20 nucleotides and most preferably between about 7 to 16 nucleotides.

Preferred PNA oligomers are those targeting a unique region in both the human and mouse PAX2 mRNA or mouse HNFα mRNA.

In another aspect the invention provides a method of synthesizing TPP-PNA conjugates according to Formula I comprising:
(a) incubating a compound of Formula II, wherein L and X are defined as above,

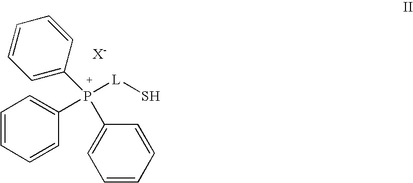

with an oxidant, to form the disulphide compound of Formula III

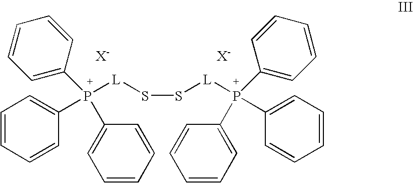

(b) reacting the compound of Formula III from step (a) with a compound of Formula IV

PNA-Z-SH IV

wherein Z and PNA are defined as above, and wherein the compound of Formula IV has been preincubated with a non-thiol containing reducing agent, to form the TPP-PNA conjugate of Formula I.

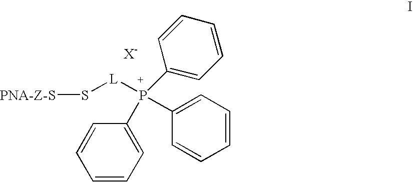

In a further aspect, the invention provides a pharmaceutical composition comprising a therapeutically effective amount of a compound of Formula I in combination with one or more pharmaceutically acceptable excipients, carriers or diluents.

In a yet further aspect, the invention provides a use of a compound of Formula I in the preparation of a medicament for the treatment of a disease or disorder that can be at least in part alleviated by antisense therapy.

The invention also provides a method of treating a patient with a disease or disorder that is susceptible to antisense therapy, which comprises the step of administering to said patient, a therapeutically effective amount of a compound of Formula I or a composition of the invention.

The invention also provides a method of diagnosing a patient with a disease or disorder that is susceptible to antisense therapy, which comprises analyzing tissues from said patient, using a compound of Formula I or a composition of the invention.

The invention also provides a method of diagnosing a patient with a disease or disorder that is susceptible to antisense therapy, comprising incubating tissues and/or blood from said patient with a compound of Formula I or a composition of the invention.

The invention further provides a method of targeting PNA oligomers to non-mitochondrial sites or organelles within a cell, including the cytoplasm and/or the nucleus, using a compound of Formula I, said method comprising delivering the PNA oligomers across the plasma membrane, without promoting selective aggregation in the mitochondria of said cell.

The invention further provides a method for modifying gene expression by administering a compound of Formula I to a cell.

The invention further provides a method for altering RNA function or processing by administering a compound of Formula I to a cell.

### DESCRIPTION OF THE DRAWINGS

In particular, a better understanding of the invention will be gained with reference to the following figures in which:

FIG. 1 is a schematic representation of uptake of a TPP-fluPNA(PAX-2) conjugate into a cell.

FIG. 2 shows the synthesis of a TPP-PNA conjugate.

FIG. 3 shows the purification and characterization of TPP-fluPNA(PAX-2) and TPP-bioPNA conjugates by RP HPLC.

FIG. 4 shows the characterization of a TPP-fluPNA(PAX-2) conjugate using a MALDI ToF mass spectroscopy analysis (4A and 4B) and immunoblotting (4C).

FIG. 5 shows uptake of a TPP-fluPNA(PAX-2) conjugate by 143B osteosarcoma (5A and 5B) and uptake of TPP-PNA conjugates by P388 cells (5C and 5D).

FIG. 6 shows the localization of a TPP-bioPNA conjugate in human fibroblasts visualized with confocal immunofluorescent microscopy. Cells were incubated with 1 μM TPP-bioPNA for 1 hr (6A and 6C) and 4 hr (6B and 6D).

FIG. 7 shows a western blot of P388 cells treated with 1 μM TPP-fluPNA(PAX2), unconjugated PNA(PAX-2) and with media only.

FIG. 8 shows the purification and characterization of TPP-fluPNA(HNF4-α) conjugates by RP HPLC (8A) and MALDI ToF mass spectroscopy analysis (8B).

FIG. 9 shows the uptake of TPP-fluPNA(HNF4-α) by mouse liver cells BNL.CL2 at 4 h (9A) and 44 h (9B). Green is TPP-fluPNA(HNF4-α) and red is MitoTracker Red CMXRos.

FIG. 10 shows RT-PCR of TPP-lysPNA(HNF4-α) conjugates showing expression of HNF4α pre mRNA splice variant induced by TPP-lysPNA(HNF4-α) transfected into BNL-CL2 liver cells using chloroquine. FIG. 10A shows lane 1: media control; lane 2: RT-PCR following addition of PNA to the cell lysis buffer during RNA isolation in cells treated with media only; lane 3: RT-PCR of HNF4-α mRNA following transfection of BNL-CL2 liver cells with the TPP-lysPNA(HNF4-α) conjugate using chloroquine. FIG. 10B shows lane 1: media control; lane 2: RT-PCR of HNF4-α mRNA following co-culture of cells for 70 hrs with 1 μM TPP-lysPNA(HNF4-α) conjugate.

### DETAILED DESCRIPTION OF THE INVENTION

In one aspect the present invention is directed towards methods of transporting PNAs into a cell using a lipophilic cation modified to dissociate from the PNA in the cytoplasm thereby preventing selective accumulation in the mitochondria.

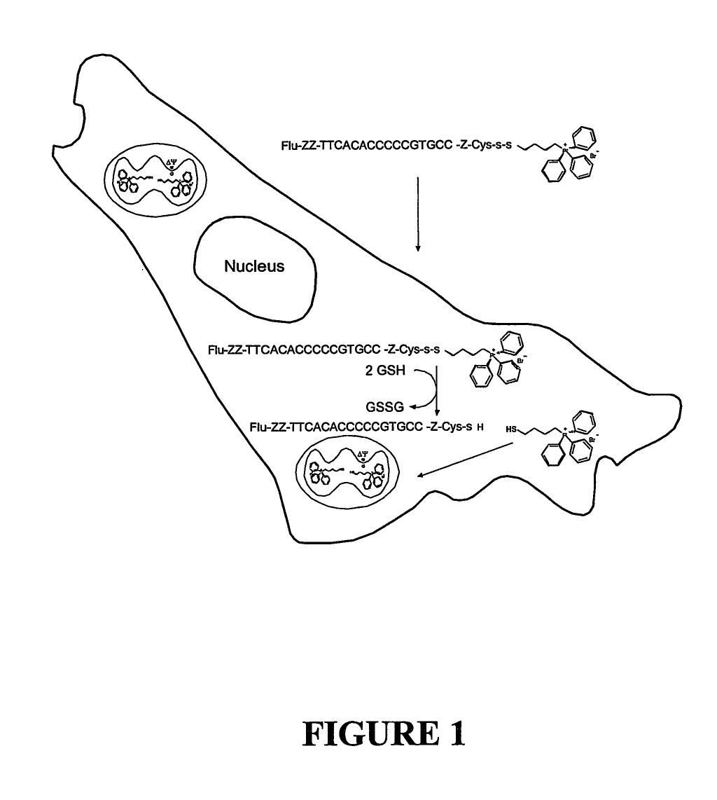

The applicants have unexpectedly found that this can be achieved by conjugating a PNA oligomer to a phosphonium cation such as thiobutyltriphenylphosphonium (TBTP) (Burns et al., 1995) via a disulfide bond that is stable in the oxidising extracellular environment but is labile in the reducing cytoplasmic milieu (FIG. 1). Once this TPP-PNA conjugate crosses the plasma membrane driven by the membrane potential, the disulfide bond is reduced by the cytoplasmic glutathione pool. The PNA is then released into the cytoplasm, while the dissociated lipophilic cation accumulates into the mitochondria. Once released, the PNA oligomer may remain in the cytoplasm or may enter the nucleus.

In one aspect the invention therefore provides a conjugate of Formula I:

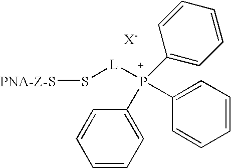

wherein L is a linker group, S-Z is a thiol-containing attachment group, X⁻ is an optional anion and PNA is a peptide nucleic acid.

The linker group L may be any chemically non-active distance-making group which joins the triphenylphosphonium cation moiety to the PNA moiety, and enables the two moieties to remain bonded together when crossing the plasma membrane.

Typically, the group will be an alkylene group. The term “alkylene” as used herein, pertains to a bidentate moiety obtained by removing two hydrogen atoms, either both from the same carbon atom, or one from each of two different carbon atoms, of a hydrocarbon compound having from 1 to 30 carbon atoms, preferably 2 to 20, more preferably 2 to 10, even more preferably 3 to 5 and most preferably 4, which may be aliphatic or alicyclic, and which may be saturated, partially unsaturated, or fully unsaturated. Thus, the term “alkylene” includes the sub-classes alkenylene, alkynylene, and cycloalkylene. The linking group may also contain one or more heteroatoms such as N, O or S.

The linking group may also be substituted by one or more substituent groups that increases the solubility of the molecule, increases the uptake of the molecule across the plasma membrane, or decreases the rate of degradation of the molecule in vivo. In particular, the linking group may be substituted by hydroxyl, thio, amino, carboxy, amido groups or groups derived from sugars or sugar derivatives.

The anion comprises a suitable inorganic or organic anion known in the art and is present when required for overall electrical neutrality. Examples of suitable inorganic anions include, but are not limited to, those derived from hydrochloric, hydrobromic, hydroiodic, sulfuric, sulfurous, nitric, nitrous, phosphoric or phosphorous acid or from an alkylsulfonic or an arylsulfonic acid. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyuvic, salicylic, stearic, succinic, sulfanilic, tartaric, toluenesulfonic, and valeric. All are generally recognized as pharmaceutically acceptable salts.

More preferably, the inorganic anions are preferred, in particular, the halo anions, especially the bromide anion.

The spacer group S-Z may be any group containing a free thiol functionality that allows the PNA to bond to the TPP moiety via a disulfide bond. Preferably, S-Z is a cysteinyl, homocysteinyl or aminothiol compound linked to 8-amino-3,6-dioxanoic acid (8-amino-3,6-dioxaoctanoic acid). Other groups that can be linked to a free thiol containing group to make a spacer group include, but are not limited to SMCC (succinimidyl 4-(N-maleimidomethyl)cyclohexane-1-carboxylate) AHEX or AHA (6-aminohexanoic acid), 4-aminobutyric acid, 4-aminocyclohexylcarboxylic acid, LCSMCC (succinimidyl 4-(N-maleimidomethyl)cyclohexane-1-carboxy-(6-amido-caproate), MBS (succinimidyl m-maleimido-benzoylate), EMCS (succinimidyl N-ε-maleimido-caproylate), SMPH (succinimidyl 6-(β-maleimido-propionamido) hexanoate, AMAS (succinimidyl N-(α-maleimido acetate), SMPB (succinimidyl 4-(p-maleimidophenyl)butyrate), β.ALA (β-alanine), PHG (Phenylglycine), ACHC (4-aminocyclohexanoic acid), β.CYPR (β-(cyclopropyl) alanine) and ADC (amino dodecanoic acid).

The phenyl groups of the triphenylphosphonium moiety may be optionally substituted with alkyl groups or any other group provided that the conjugate remains hydrophobic enough to transfer across the cell membrane.

PNAs are described in WO 92/20702 and WO 92/20703, the contents of which are hereby incorporated by reference. The PNA moiety for use in the conjugates of the invention comprises a PNA oligomer which is complementary to at least one target nucleotide sequence. The PNA may have exact sequence complementarity to the target sequence, or only partial complementarity, provided that the resulting hybridised duplex structure is sufficiently stable to block or inhibit translation or transcription of the target sequence. The target nucleotide sequence may comprise any type of cellular nucleic acid material including but not limited to DNA, mRNA, tRNA, rRNA, SnRNA and microRNA.

The PNA moiety may incorporate one or more amino acids. For example, the addition of lysine to the PNA oligomer (Lys-PNA) may increase the solubility of the oligomer.

The binding of a PNA strand to a DNA or RNA strand can occur in either the parallel or antiparallel orientation. As used in the present invention, the term complementary as applied to PNA does not specify a particular orientation, although it is noted that the most stable orientation of PNA/DNA and PNA/RNA is anti-parallel. In a preferred embodiment, PNA targeted to single stranded RNA is complementary in an anti-parallel orientation.

In a preferred embodiment, the PNA moiety contains between about 3 to 25 nucleotides, preferably between about 5 to 20 nucleotides and most preferably between about 7 to 16 nucleotides.

PNA oligomers can be obtained commercially, for example, from Pantheco A/S (Horsholm, Denmark). The oligomers may be obtained as N-protected PNAs such as tBoc-PNA and FmocPNA (Celera Diagnostics, Alameda, Calif.), or as labelled oligomers such as rhodamine-PNA, maleimide-PNA, biotin-PNA and fluorescein-PNA (Gene Therapy Systems, Inc, San Diego, Calif.).

Preferred PNA oligomers are those targeting a unique region in both the human and mouse PAX2 mRNA and those targeting mouse HNF4α. Other preferred PNA oligomers are those targeting n-myc (Sun, L., Fuselier, J. A., Murphy, W. A., and Coy, D. Peptides, 23: 1557-1565, 2002), c-myc (Cutrona, G. et al, Nat Biotechnol, 18: 300-303, 2000), bcl-2 (Mologni, L., Nielsen, P. E., and Gambacorti-Passerini, C. Biochem Biophys Res Commun, 264: 537-543, 1999), N-myc (Pession, A., Tonelli, R., Fronza, R., Sciamanna, E., Corradini, R., Sforza, S., Tedeschi, T., Marchelli, R., Montanaro, L., Camerin, C., Franzoni, M., and Paolucci, G. Int J Oncol, 24: 265-272, 2004) and PIM-1 (Bertrand, J. R, Sumbatyan, N., and Malvy, C. Nucleosides Nucleotides Nucleic Acids, 22: 1611-1613, 2003). These documents are herein incorporated by reference.

The optional reporter molecule enables the conjugate to be easily detected with high sensitivity, and is compatible with the biological function of the conjugate. Such moieties include but are not restricted to a range of molecular tags such as affinity labels (for example, biotin, streptavidin, rhodamine, maleimide and the like) that can be attached to PNAs or more preferably fluorophores (such as for example Cy3, Cy5 and Cy2), or most preferably, fluoroscein. Reporter gene literature is reviewed in Herrera-Estrella et al., Nature 303: 209-213, 1993, and Handbook of Fluorescent Probes and Research Products, Ninth Ed., Richard P. Haugland, Molecular Probes, 2002.

The presence of a reporter molecule allows the sensitive detection of the conjugates. For example, biotinylation transforms a poorly detectable molecule into a conjugate that can be probed for using streptavidin. Conjugation of flurophores such as fluorescein allows the use of confocal laser scanning fluorescence microscopy (Three-Dimensional Confocal Microscopy, Stevens, J. K.; Mills, L. R.; Trogadis, J. E.; Eds, pp 101-129, 1994).

PNA oligomers obtained commercially may already incorporate the spacer group S-Z at the 3′ end, and/or other spacer groups at the 5′ end of the oligomer as required for reporter molecules. The spacer groups at the 5′ end may be 8-amino-3,6-dioxanoic acid or any other suitable spacer group. For example, PNA oligomers incorporating reporter groups biotin and fluoroscein may be obtained from Applied Biosystems Inc (Bedford, Mass.).

Detection of the conjugates may be important in some applications such as trace localization/distribution in an organism/tissue, for example, to assess whether the conjugate crosses the blood-brain barrier or the placenta. In other applications, for example, treatment of disease, the reporter molecule may not be necessary.

The conjugates of the invention may also be modified for the transport of other modified anti-sense agents such as locked nucleic acid derivatives (LNAs), gripNAs™ and morpholino analogues.

LNAs are comprised of ribonucleotide monomers having a 2′,4′-bridge and are described in International PCT publication WO 99/14226 (incorporated herein by reference). Examples of LNAs include amino-LNA, thio-LNA, seleno-LNA, methylene-LNA and oxy-LNA. LNAs may be prepared using techniques known in the art, for example, as described in International PCT publication WO 03/09546.

Morpholino oligomers are described in International PCT publication WO 98/32467 (incorporated herein by reference) and comprise morpholino subunits linked together by, for example, uncharged, phosphorous-containing linkages, one to three atoms long, joining the morpholino nitrogen of one subunit to the 5′ exocyclic carbon of an adjacent subunit. Linked to each subunit is a purine or pyrimidine base-pairing moiety effective to bind, by base-specific hydrogen bonding, to a base in a target polynucleotide.

Other PNAs that can be transported into the cell using the conjugates of the invention include PNA oligomers wherein the backbone has been stabilised by the introduction of a prolyl unit (D'Costa et al, 1999, incorporated herein by reference) or gripNAs™ (see www.activemotif.com, also incorporated herein by reference) which are comprised of a backbone of alternating HypNA and pPNA monomers, with the bases attached through methylene carbonyl linkages (Efimor, V. A. et al. (1998) NAR 26, 566-575). Other backbone stabilised PNA derivatives include aminoethylprolyl-PNA and aminoethylpyrrolidine-PNA.

In another aspect the invention provides a method of synthesizing TPP-PNA conjugates according to Formula I comprising:
(a) incubating a compound of Formula II, wherein L and X are defined as above,

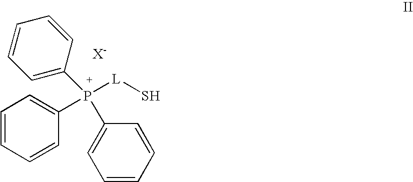

with an oxidant, to form the disulfide compound of Formula III

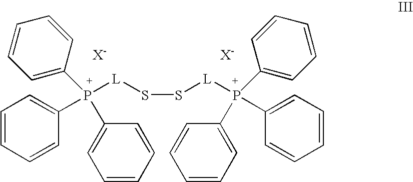

(b) reacting the compound of Formula III from step (a) with a compound of Formula IV

HS-Z-PNA IV

wherein Z and PNA are defined as above, and wherein the compound of Formula IV has been pre-incubated with a non-thiol containing reducing agent, to form the TPP-PNA conjugate of Formula I.

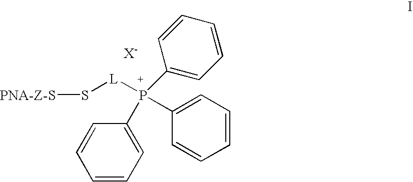

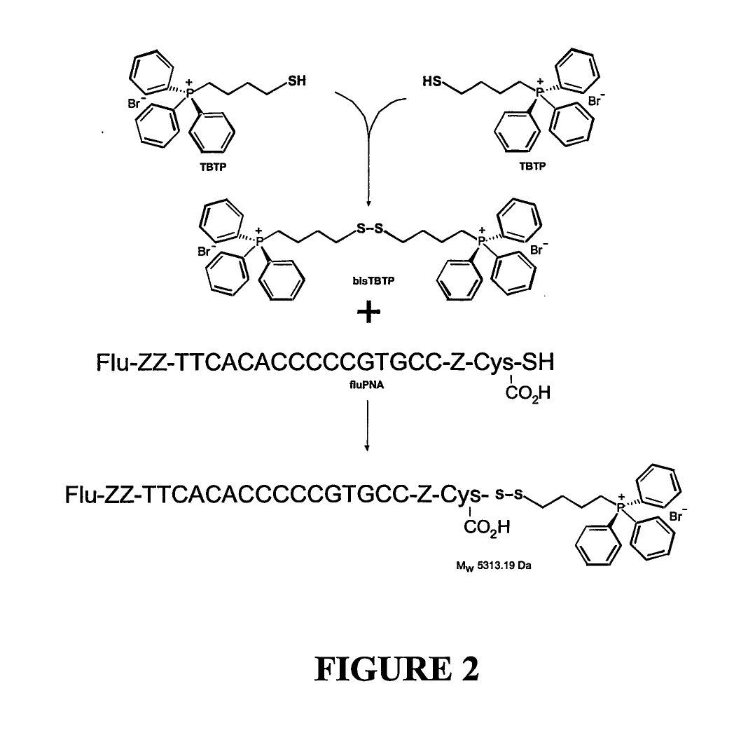

As described above, the synthesis of the TPP-PNA conjugate can be carried out using a two stage thiol disulfide exchange mechanism, also illustrated by the example shown in FIG. 2. The first stage is the synthesis of a bistriphenylphosphonium disulfide (bisL-TTP) (Scheme 1).

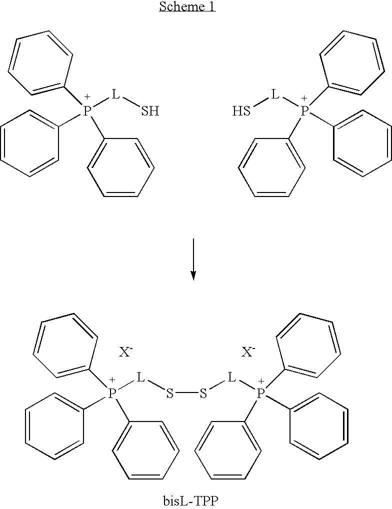

The HS-L-TPP compound is generated by base hydrolysis of acylated thiol-TPP, for example, by incubation with NaOH.

### In the second stage the bisL-TPP is reacted with a thiol-containing PNA oligomer to form the TPP-PNA conjugate (Scheme 2).

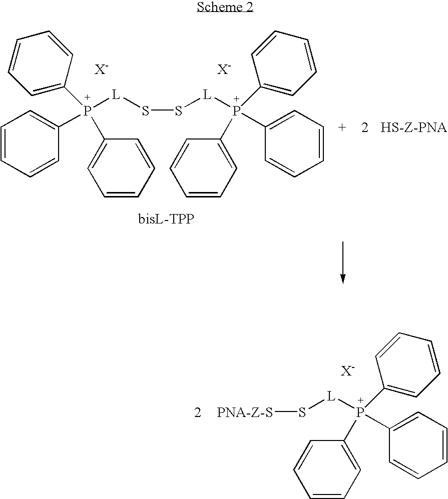

The PNA oligomer is first preincubated with a non-thiol containing reducing agent such as Tris[2-carboxyethyl]phosphine hydrochloride.

The cysteine residue of the thiol-linked PNA conjugate forms a disulfide linkage with the thiol-linked TPP to make a TPP-PNA conjugate. The coupling efficiency of the thiol containing compounds can be monitored by assaying the free thiol groups.

In a preferred embodiment the compound of formula II is thiobutyltriphenylphosphonium (TBTP). The lipophilic cation TBTP is a thiol reagent that is selectively directed to the mitochondrial matrix driven by the membrane potential (FIG. 2). TBTP has a lipophilic core, and a four carbon chain at the end of which is a thiol group to enable thiol-disulfide exchange.

Commercially available TBTP includes a protecting acyl group on the thiol to prevent oxidation during synthesis and storage. After deprotection of the acyl-TBTP by base hydrolysis the solution is adjusted to neutral pH and treated with a thiol-oxidising agent. Preferably, the thiol-oxidising agent is diamide. Diamide stoichiometrically oxidises thiols to disulfides by the following reaction:

(CH₃)₂NCON═NCON(CH₃)₂+2TBTP [symbol] (CH₃)₂NCONHNHCON(CH₃)₂+bisTBTP (1)

The reaction is driven forward by an excess of TBTP, which is inexpensive and easily obtained. One of the advantages of this synthetic strategy is that the only major chemical species that are present at the end of the reaction are the TPP-PNA product and an excess of unreacted bisL-TPP. Purification of the reaction products by RP-HPLC allows the valuable unconjugated PNA to be recovered and re-used.

In addition to the thiol disulfide exchange method described above (see Schemes 1 and 2), the synthesis of the TPP-PNA conjugates can also be carried out by oxidation of a mixture of thiols.

This method uses the disulfide (bisL-TPP), which is reduced to thiol in situ, and then oxidized with H₂O₂ in the presence of PNA-Z-SH, as indicated in the following reactions.

BisL-TPP+TCEP+H₂O→2TPP-L-SH+TCEP.O

PNA-SH+TPP-L-SH+H₂O₂→PNA-S—S-L-TPP+2H₂O

In another aspect, the invention provides a pharmaceutical composition comprising a therapeutically effective amount of a compound of formula I in combination with one or more pharmaceutically acceptable excipients, carriers or diluents.

Suitable excipients, carriers and diluents can be found in standard pharmaceutical texts. See, for example, Handbook for Pharmaceutical Additives, 2^{nd} Edition (eds. M. Ash and I. Ash), 2001 (Synapse Information Resources, Inc., Endicott, N.Y., USA) and Remington's Pharmaceutical Science, (ed. A. L. Gennaro) 2000 (Lippincott, Williams and Wilkins, Philadelphia, USA) which are incorporated herein by reference.

Excipients for use in the compositions of the invention include, but are not limited to microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

Pharmaceutical carriers include solid diluents or fillers, sterile aqueous media and various non-toxic organic solvents, and the like.

The term “pharmaceutically acceptable” as used herein pertains to compounds, ingredients, materials, compositions, dosage forms and the like, which are within the scope of sound medical judgment, suitable for use in contact with the tissues of the subject in question (e.g. human) without excessive toxicity, irritation, allergic response, or other problem or complication, commensurate with a reasonable benefit/risk ratio. Each carrier, diluent, exipient, etc., must also be “acceptable” in the sense of being compatible with the other ingredients of the formulation.

The pharmaceutical compositions of the invention may be combined with other active pharmaceutical compounds such as anti-cancer agents, anti-inflammatory agents, anti-viral agents such as anti-HIV agents, anti-bacterial agents and the like.

In a further aspect, the invention provides a use of a compound of formula I in the preparation of a medicament for the treatment of a disease or disorder that can be, at least in part, alleviated by antisense therapy.

The medicament may be prepared by any methods well known in the art of pharmacy. Such methods include the step of bringing into association the active compound with a carrier, diluent, exipient or combination thereof, as discussed above.

In a yet further aspect, the invention provides a method of treating a patient with a disease or disorder that is susceptible to antisense therapy, which comprises the step of administering to said patient, a therapeutically effective amount of a compound of formula I or a composition of the invention.

Diseases or disorders that are susceptible to antisense therapy include, but are not limited to, bacterial and viral infections, cancer, metabolic diseases and immunological disorders.

In particular, the conjugates of the invention can be used to treat the following diseases for which antisense therapy clinical trials are in progress: HIV infection and hepatitis C infection; cancers such as melanoma, pancreatic adnocarcinoma, actue myeloid leukemia, myeloma, small cell lung cancer, prostate cancer, ovarian carcinoma, breast cancer, glioma; metabolic diseases such as hypercholesterolemia; and immunological disorders such as amyloid light chain amyloidosis.

The conjugates or pharmaceutical compositions of the invention can be administered via oral, parenteral (such as subcutaneous, intravenous, intramuscular, intrasisternal and infusion techniques), rectal, intranasal or topical routes. In general, these compounds are administered in doses ranging from about 0.5 to about 500 mg per day, in single or divided doses (such as from 1 to 4 doses per day).

It will be appreciated by one of skill in the art that appropriate dosages of the compounds, and compositions comprising the compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition and the general health and prior medical history of the patient.

The active compounds of this invention can be administered alone or in combination with pharmaceutically acceptable excipients, carriers or diluents by any of the routes previously indicated, and such administration may be carried out in single or multiple doses.

More particularly, the novel therapeutic agents of this invention can be administered in a wide variety of different dosage forms, they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions injectable solutions, elixirs, syrups, and the like. Such carriers include solid diluents or fillers, sterile aqueous media and various non-toxic organic solvents, and the like.

Moreover, oral pharmaceutical compositions can be suitably sweetened and/or flavoured. In general, the conjugates of the invention are present in such dosage forms at concentration levels ranging from about 5.0% to about 70% by weight.

For oral use in treating the various disorders and conditions referred to above, the conjugates can be administered, for example, in the form of tablets or capsules, or as an aqueous solution or suspension. Tablets may containing various excipients such as described above.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-muscular and subcutaneous injection purposes. The preparation of all these solutions under sterile conditions is readily accomplished by standard pharmaceutical techniques well known to those skilled in the art.

For intramuscular, parenteral and intravenous use, sterile solutions of the active ingredient can be prepared, and the pH of the solutions should be suitably adjusted and buffered. For intravenous use, the total concentration of salutes should be controlled to render the preparation isotonic.

The term “patient” as used herein refers to a human or non-human mammal. Examples of non-human mammals include livestock animals such as sheep, cows, pigs, goats, rabbits, deer, ostriches and emus; and companion animals such as cats, dogs, rodents, and horses.

The term “treatment” as used herein in the context of treating a condition, pertains generally to treatment and therapy, whether of human or animal, in which some desired therapeutic effect is achieved, for example, the inhibition of progress of the condition, and includes a reduction in the rate of progress, a halt in the rate of progress, amelioration of the condition, and cure of the condition. Treatment as a prophylactic measure (i.e., prophylaxis) is also included.

“Treatment” also includes combination treatments and therapies, in which two or more treatments or therapies are combined, for example, sequentially or simultaneously.

For example, a therapeutically effective amount of a compound of formula I could be combined with or used in conjunction with radiation therapy or chemotherapy in the treatment of cancer.

The term “therapeutically-effective amount” as used herein, pertains to that amount of an active compound, or a material, composition or dosage form comprising an active compound, which is effective for producing some desired therapeutic or prophylactic effect, commensurate with a reasonable benefit/risk ratio.

The conjugates of the invention are not limited to use in mammals and may be used to target nucleotide oligomers in any living organism, including unicellular prokaryotic and eukaryotic organisms to multicellular eukaryotic organisms. The conjugates of the invention may also have applications in the biotechnology field, for example, for limiting alcohol production in yeast.

The invention also provides a method of diagnosing a patient with a disease or disorder that is susceptible to antisense therapy, which comprises analyzing tissues from said patient, using a compound of Formula I or a composition of the invention.

The invention also provides a method of diagnosing a patient with a disease or disorder that is susceptible to antisense therapy, comprising incubating tissues and/or blood from said patient with a compound of Formula I or a composition of the invention.

The conjugates of the invention may be used in both in vivo and in vitro methods. For example, cells removed from a patient may be treated with the conjugates of the invention in vitro and then returned to the patient.

The invention further provides a method of targeting PNA oligomers to non-mitochondrial sites or organelles within a cell, including the cytoplasm and/or the nucleus, using a compound of Formula I, said method comprising delivering the PNA oligomers across the plasma membrane, without promoting selective aggregation in the mitochondria of said cell.

The invention further provides a method for modifying gene expression by administering a compound of Formula I to a cell.

The invention further provides a method for altering RNA function or processing by administering a compound of Formula I to a cell.

The conjugates of the invention utilise a lipophilic cation modified to dissociate from the PNA to transport PNA oligomers into the cell. Dissociation occurs in the cytoplasm thereby preventing selective accumulation in the mitochondria

The PNA oligomers delivered to the cell by the conjugates of the invention may also localize to the nucleus to affect pre-mRNA processing, or other RNA function in the nucleus. For example, small nuclear RNA (SnRNA), rRNA and miRNA may also be targeted.

The invention will now be described in more detail with reference to the following non-limiting experimental section.

### EXAMPLE 1

### Methods

### Chemical Synthesis of Bisthiobutyltriphenylphosphonium (bisTBTP)

Thiobutyltriphenylphosphonium (TBTP) was generated by base hydrolysis of acylated TBTP as described (Burns et al., 1995). Equal volumes of 1 M NaOH and 500 mg acylated TBTP dissolved in 95% ethanol were mixed and incubated for 20 minutes at room temperature, then diluted (1:40) in 150 mM HEPES, pH 7.3. The solution of TBTP at pH 7.3 was incubated with 0.2 g diamide ((CH₃)₂NCON═NCON(CH₃)₂, Sigma) for 1 h at room temperature. The formation of bisTBTP was followed by the disappearance of free thiols assayed as described in the thiol assay section below. After quenching with 1 M HCl (0.5 vol.), 0.5 g NaBr was added to ensure a Br⁻ counterion. The bisTBTP was extracted into 1 vol. dichloromethane three times, leaving unreacted diamide in the aqueous phase. The bisTBTP was precipitated from the dichloromethane by addition of diethyl ether (50 mL) giving a white powder (244 mg, 48% yield). The identity of bisTBTP was determined by ¹H NMR spectroscopy in CDCl₃, acquired using a Varian Gemini 200 MHz spectrophotometer at 25° C.

The analysis gave the following peaks: δ 7.6-8.0 (30 H, m, (Ph₃P—CH₂—CH₂—CH₂—CH₂)₂S₂), δ 3.92 (4H, b, (Ph₃P—CH₂—CH₂—CH₂—CH₂)₂S₂), δ 2.80 (4H, t, J=7.2 H₂ (Ph₃P—CH₂—CH₂—CH₂—CH₂)₂S₂), δ 2.06-2.11 (4H, m, (Ph₃P—CH₂—CH₂—CH₂—CH₂)₂S₂), δ 2.09 (4H, m, (Ph₃P—CH₂—CH₂—CH₂—CH₂)₂S₂) δ 1.74 (4H, m, (Ph₃P—CH₂—CH₂—CH₂—CH₂)₂S₂). Trace amounts of impurity detected by NMR had chemical shifts corresponding to diethyl ether (δ 1.19-1.25, 3.47 and 3.49). The final product was stored at −20° C. The identity of bisTBTP was further confirmed by base hydrolysis with 1 M NaOH which exposed 1.9 mol sulfhydryl per mol bisTBTP quantitated by the free thiol assay described below.

### Thiol Assay

The coupling efficiency of thiol containing compounds was monitored by assaying free thiol groups. Thiol groups react with 5,5′-dithiobis(2-nitrobenzoic acid) (DTNB) to form 5-thio-2-nitrobenzoic acid (TNB) that has a strong absorbance at 412 nm (ε₄₁₂=13.6×10³ M⁻¹ cm⁻¹; (Ellman and Lysko, 1979)). The stoichiometry of the reaction is 1:1 and therefore the formation of TNB is proportional to the free thiol concentration. The total thiol content was estimated by adding 20 μL sample to 980 μL buffer containing 80 mM NaHPO₄ (pH 8.0, NaOH) and A₄₁₂ was read before initiating the reaction by addition of 20 μL DTNB (10 mM DTNB in 0.1 M NaHPO₄, pH 8.0) and again 20 minutes later and the difference in absorbance used to calculate the thiol content. To correct for the absorbance due to degraded DTNB, the A₄₁₂ of a buffer sample to which 20 μL DTNB was added was subtracted from the final absorbance.

### Chemical Synthesis and Purification of Disulfide Linked TPP-bioPNA Conjugates

Peptide nucleic acids (PNA) were synthesised by Applied Biosystems Inc. (Bedford, Mass.). The oligomers used were: Fluoroscein-XX-TTCACACCCCCGTGCC-X-Cys-CO₂H and Biotin-XX-GTTGGCTCTCT-X-Cys-CO₂H, where X is 8-amino-3,6-dioxanoic acid. Fluoroscein-XX-TTCACACCCCCGTGCC-X-Cys-CO₂H targets a unique region in both the human and mouse PAX2 mRNA (Acc. No. NM_{—}003989.1 and X55781.1, respectively).

To conjugate these PNAs to TBTP, the PNA oligomers (50 nmol) in 50 μL 10 mM HEPES, 1 mM EDTA, pH 7.5 were incubated with a non-thiol containing reducing agent, Tris[2-carboxyethyl]phosphine hydrochloride (TCEP.HCl), 2 equiv., at 37° C. for 1 h. Then bisTBTP (5 equiv.) in 20 μL 10 mM HEPES, 1 mM EDTA, pH 7.5 was added and incubated at 37° C. for a further 4 h. The reaction products were separated by RP-HPLC on a C₄ analytical column (Vydac, 300 Å, 4.6 mm×250 mm), using a Waters 450 HPLC system and a linear gradient from 0.1% TFA in water to 90% acetonitrile and 0.1% TFA was run over 30 minutes. Disulfide linked TPP-bioPNA conjugate peaks were detected by absorbance at 260 nm, collected, lyophilised and dissolved in water for further analysis.

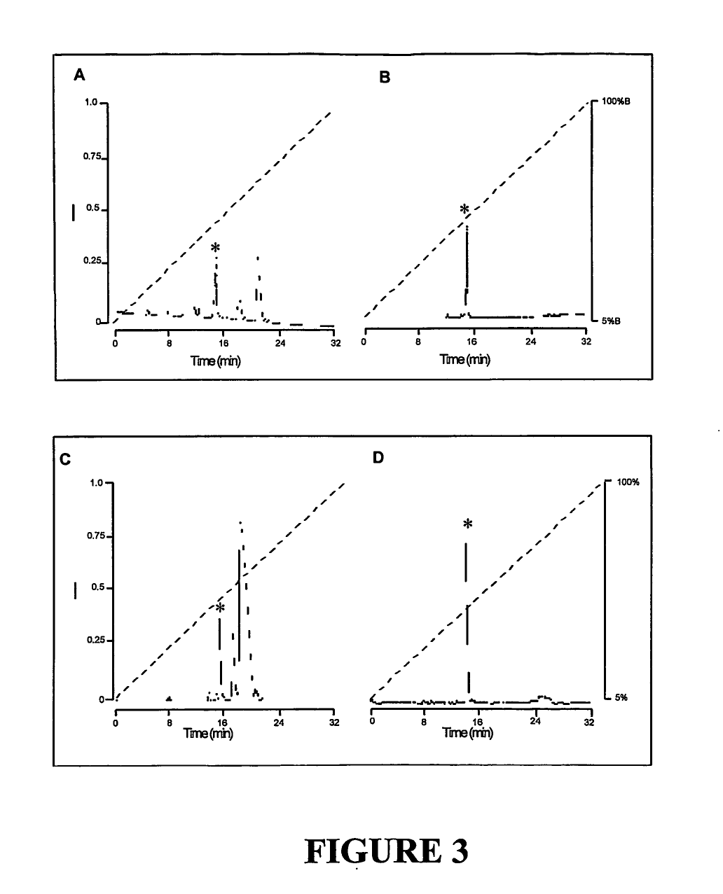

The major peak at ˜16 min (shown by an asterisk in FIG. 3A), due to the TPP-bioPNA conjugate, was collected, lyophilized, dissolved in water and a sample analysed by RP-HPLC (FIG. 3B). FIG. 3C shows purification of TPP-fluPNA by RP-HPLC. The peak at ˜16 min (shown by an asterisk) is that of the TPP-fluPNA conjugate and this was collected and a sample analysed by RP-HPLC (FIG. 3D).

The concentration of the TPP-PNA conjugates was determined at 55° C. using the cumulative extinction coefficients of unmodified PNA (97,900 M⁻¹.cm⁻¹) and TBTP (2,500 M⁻¹.cm⁻¹), to give a value of 100,400 M⁻¹.cm⁻¹. The extinction coefficient for TPP-fluPNA is 158,500 M⁻¹.cm⁻¹.

### MALDI ToF Mass Spectrometry

PNA conjugates (˜0.5 pmol) in water were mixed with 3,5-dimethoxy-4-hydroxycinnamic acid (˜0.5 μL of a 10 mg.mL⁻¹ solution) and after crystalisation were analysed by Matrix Assisted Laser Desorption/Ionization Time of Flight Mass Spectrometry (MALDI ToF MS) using a Finnigan MAT Lasermat 2000 instrument. Spectra were acquired in positive ion mode using melittin (M_{w} 2,846 Da) as an external mass calibrant.

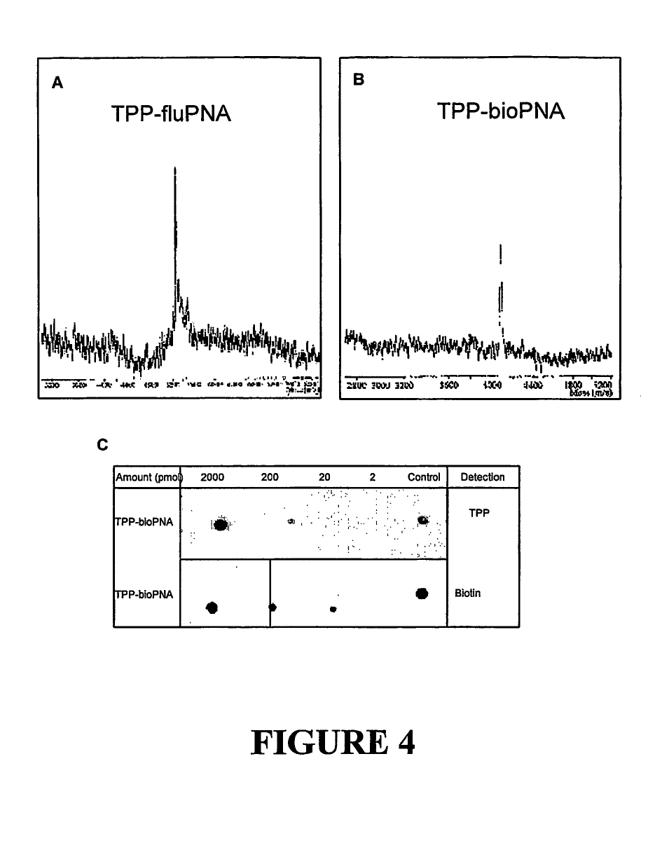

MALDI ToF analysis of purified TPP-PNAs is shown in FIGS. 4A and 4B. The observed mass for the TPP-bioPNA was 4090.7 Da, within 0.01% of the calculated mass (4091.8 Da), as expected for external mass calibration (FIG. 4A). The observed mass for the TPP-fluPNA was 5313.19 DA, within 0.1% of the calculated mass (4962.74 DA) (FIG. 4B).

### Cell Culture and Incubations With PNA

Cells (143B, human fibroblasts, COS-7, P388 and IMCD) were grown at 37° C. and 5% CO₂ in humidified atmosphere in Dulbecco's modified Eagle's medium supplemented with 10% inactivated foetal calf serum (FCS). All cell culture media contained 100 units.mL⁻¹ penicillin, and 100 μg.mL⁻¹ streptomycin. Protein was quantitated by the bicinchoninic acid assay. Cells were incubated with 1 μM TPP-PNA or with PNA alone for the indicated times. The disulfide bond in the TPP-PNA is stable in the oxidizing extracellular environment but is labile in the reducing cytoplasmic mulieu (FIG. 1).

### 143B Cell Incubations

For incubations in suspension 143B cells were harvested using trypsin and 10⁶ cells were suspended in 1 mL DMEM, 10 mM HEPES, pH 7.0 and 10% FCS. For cell subfractionation, 143B cells were grown to confluence in 24 well tissue culture plates overnight and then incubated with 1 μM TPP-PNA conjugates ±10 μM FCCP for 1 h at 37° C. and after washing the cells were harvested by scraping in 250 mM sucrose, 20 mM MOPS, 3 mM EDTA, pH 6.7, and 1 mg.mL⁻¹ digitonin. A mitochondria enriched fraction was prepared from 200 μL crude suspension by centrifugation (10,000×g, 1 min) through 300 μL oil (58% silicone oil (Dow Corning)/42% dioctyl phthalate) into 100 μL 0.5 M sucrose/0.1% Triton X-100, leaving a cytoplasm enriched upper layer. About 92-96% of total citrate synthase (Srere, 1969) and 0.3-1% lactate dehydrogenase activities (Berry et al., 1991) were found in the mitochondria enriched fraction. Both fractions were immunoblotted to detect TPP-PNA conjugate localisation.

### Gel Electrophoresis and Immunoblotting

Immunoblotting of TPP-PNA conjugates is shown in FIG. 4C. Serial dilutions of the TPP-PNA conjugates were absorbed on nitrocellulose and the triphenylphosphonium moiety detected using anti triphenylphosphonium serum. BSA conjugated to IBTP (˜1 μg protein) was used as a positive control. Horse radish peroxidase conjugated to extravidin was used to detect biotin and the bioPNA oligomer (˜5 nmol) was used as a positive control.

For conjugate detection, 143B cell lysates treated with TPP-PNA conjugates (˜5 nmol) in 20 μL loading buffer (50 mM Tris, 4% SDS, 12% glycerol, 2% 2-mercaptoethanol, 0.01% coomassie brilliant blue) were separated on 18.5% Tris-tricine gels using a BioRad Mini Protean system (Schagger and von Jagow, 1987). For PAX-2 detection, P388 and IMCD cell lysates in loading buffer were resolved on 12.5% Tris-glycine gels (Laemmli, 1970). Gels were then either fixed and stained with coomassie brilliant blue [0.1% (w/v) coomassie brilliant blue R-250, 45% (v/v) methanol and 10% (v/v) acetic acid], or electrotransfered onto 0.2 μm nitrocellulose using a BioRad Mini Trans-Blot system (100 V, 1 h) in transfer buffer (25 mM Tris-HCl, pH 8.3, 192 mM glycine, 20% methanol) and then blocked with 2% (w/v) fat-free milk powder in TBS (5 mM Tris.HCl, pH 7.4, 20 mM NaCl), 0.1% Tween-20. Rabbit anti-PAX2 polyclonal antibody (Zymed) diluted 1:1000 in TBS, 0.1% fat free milk powder, 0.1% Tween-20 was used for PAX-2 detection following overnight incubation with the membrane. After 3×10 min washes in TBS, 0.1% Tween-20, horseradish peroxidase conjugated goat antirabbit IgG (1:10,000, Biorad) was used as a secondary antibody. To detect biotin, horse radish peroxidase conjugated extravidin (1:3,000, Sigma) was used. In both cases secondary antibody binding was carried out for 1 h at room temperature, followed by 3×10 min washes in TBS and visualized by chemiluminescence using a Pierce Super Signal R chemiluminescence substrate with Kodak X-OMAT™ AR imaging film.

### Fluorescence Microscopy

For immunocytochemistry trypsinised human fibroblasts (5,000 cells per well) were plated onto 13 mm diameter glass coverslips in 24 well plates overnight. Following incubation for 4 h at 37° C. with 1 μM TPP-PNA conjugates, cells were fixed with 4% paraformaldehyde (PFH) in TBS for 30 min, washed with TBS and incubated with 10% FCS/0.1% Triton X-100/TBS (TBST) for 10 min. The IgG fraction of anti-triphenylphosphonium serum (1:500) was diluted in TBST, then added and incubated overnight at 4° C. The IgG fraction of preimmune serum was used as a control. After washing with TBS (3×5 min) the cells were incubated with anti-rabbit IgG Oregon green fluorophore-conjugated secondary antibody diluted 1:100 in TBS for 15 min in the dark. The cells were washed in TBS and incubated for another 15 min with streptavidin conjugated CY3 (1:200, Molecular Probes) in the dark, to detect biotin. Cells were then washed in TBS, mounted in DABCO/PVA medium (15 g PVA, 15 g 1,4-diazabicyclo (2,2,2) octane in 30% glucerol in 0.1 M Tris, pH 8.5) and mounted onto coverslips. Images were acquired using a BioRad MRC 600 laser-scanning confocal microscope using a Nikon Diaphot TMD inverted microscope and Nikon ×60 NA 1.4 oil immersion Plan-Apochromat objective. The 568 nm and 488 nm lines of a Krypton-Argon laser and K1/K2 filter blocks were used at identical gain, black settings and time frame.

For real time fluorescence microscopy cells were grown in 35 mm diameter dishes overnight until they reached 80% confluence. Following incubation for 1 h at 37° C. with 1 μM disulfide linked TPP-fluPNA conjugates the cells were incubated with 25 nM concentrations of MitoTracker Red (Molecular Probes) for 30 min at 37° C. The cells were then washed in TBS (3×10 min) and fresh DMEM media was added. Images were acquired using a Zeiss inverted confocal microscope with a Plan-Neofluar 40×/1.3 oil DIC objective, line 488 nm and Zeiss Imaging Software with equal exposure times.

### Results

### Synthesis and Characterisation of TPP-PNA Conjugates

The synthesis of the TPP-PNA conjugate was carried out in two stages as outlined in FIG. 2. The first stage was the synthesis of bisthiobutyl-triphenylphosphonium (bisTBTP) that would then link to thiol-containing compounds by a disulfide bond to a triphenylphosphonium cation. In the second stage the bisTBTP was reacted with a thiol containing PNA oligomers to form the TPP-PNA conjugate.

TBTP has a protecting acyl group on the thiol to prevent oxidation during synthesis and storage. After deprotection of the acyl-TBTP by base hydrolysis the solution was adjusted to neutral pH and treated with the thiol-oxidising agent diamide. To synthesise bisthiobutyltriphenylphosphonium (bisTBTP) from monomeric TBTP, diamide stoichiometrically oxidises thiols to disulfides by the following reaction:

(CH₃)₂NCON═NCON(CH₃)₂+2TBTP [symbol] (CH₃)₂NCONHNHCON(CH₃)₂+bisTBTP (1)

Diamide oxidised TBTP to bisTBTP within 30 seconds as seen by the rapid disappearance of free thiols on addition of diamide (data not shown). The reaction was left for an hour to ensure complete oxidation and the reaction was then quenched with acid. The bisTBTP was extracted from the diamide with dichloromethane and precipitated as a white powdery solid with a yield of about 50%. The identity of bisTBTP was confirmed by ¹H NMR The shift in resonance of the peaks of 4 H are diagnostic of protons adjacent to a disulfide bond and indicated the presence of a disulfide link that was absent in TBTP. Base treatment of bisTBTP completely hydrolysed the disulfide bond releasing 1.90-2.0 mol free TBTP per mol of bisTBTP as detected by the thiol assay, and free thiols were not detected in bisTBTP (data not shown).

The TPP-PNA conjugates were purified by RP-HPLC and analysed by immunoblotting against the phosphonium moiety and against the biotin tag on one of the PNA oligomers. The identity of the TPP-PNA conjugates was confirmed by MALDI ToF mass spectrometry. To show that there were disulfide bonds present in both bisTBTP and in TPP-PNA, the compounds were base hydrolysed and the resulting free thiols quantitated (data not shown). The delivery of the PNA oligomer to the cytoplasm using the TPP-PNA conjugate was determined by western blotting and immunofluorescent confocal microscopy.

The PNA sequence was designed to target a unique region in both the human and mouse PAX-2 mRNA and had a cysteine amino acid at the 3′ end and a fluoroscein tag at the 5′ end. A second PNA had a cysteine amino acid at the 3′ end and a biotin tag at the 5′ end. The triphenylphosphonium and biotin moieties enabled their detection by immunohistochemistry using either an anti-phosphonium antibody, or a streptavidin-linked enzyme, respectively. The fluoroscein tag was used to follow TPP-PNA uptake in live cells by fluorescence microscopy. The disulfide linked TPP-PNA conjugates were formed through a disulfide exchange reaction of the free thiol from the cysteine with bisTBTP. The TPP-PNA conjugate was formed within an hour at pH 7.5.

### Purification and Characterisation of the TPP-PNA Conjugate

The TPP-PNA conjugates were purified by RP HPLC (FIG. 3). The hydrophobic nature of the triphenylphosphonium moiety caused the conjugate to bind with higher affinity to the stationary phase, and to elute later than the unmodified PNA. The excess TBTP eluted latest due to its hydrophobicity (FIGS. 3 A and C). A second RP-HPLC run of the TPP-PNA fraction under the same conditions gave a single peak confirming the purity of the conjugate (FIGS. 3 B and D). The identity of the conjugates was confirmed by MALDI ToF mass spectrometry which gave the expected molecular weights (FIGS. 4 A and B). The composition of the TPP-PNAs was further characterised by immunoblotting (FIG. 4 C). The conjugates were adsorbed onto nitrocellulose, and probed for the triphenylphosphonium moiety using the cognate antiserum (FIG. 3 G). The biotin prosthetic group was detected by streptavidin binding conjugated to a horse radish peroxidase enzyme (FIG. 4 C). The antibody and the HRP enzyme could detect low amounts of the TPP-PNA conjugate (FIG. 4 C).

In summary, the TPP-PNA conjugates were synthesised, purified by RP HPLC and characterised by mass spectrometry and immunoblotting. This technique can be applied easily to other PNA oligomers to form a range of triphenylphosphonium-linked PNA conjugates.

### Uptake of TPP-PNA By Cells

Two techniques were used to analyze the efficiency of delivery of the TPP-PNA conjugates to the cytoplasm of cells in culture. The first was western blotting of mitochondrial and cytosolic fractions separated from conjugate-treated cells by homogenation followed by centrifugation. The second technique, fluorescent microscopy, used a fluorophore-conjugated antibody specific for the anti-triphenylphosphonium antibody and a streptavidin-linked fluorophore to detect the biotin tag on the PNA. In addition live cells incubated with the fluorosceinPNA and stained with a mitochondria-specific dye were used to follow the uptake and distribution of the PAX2 PNA.

### Western Blotting Following Incubation of TPP-PNA With 143B and P388 Cells

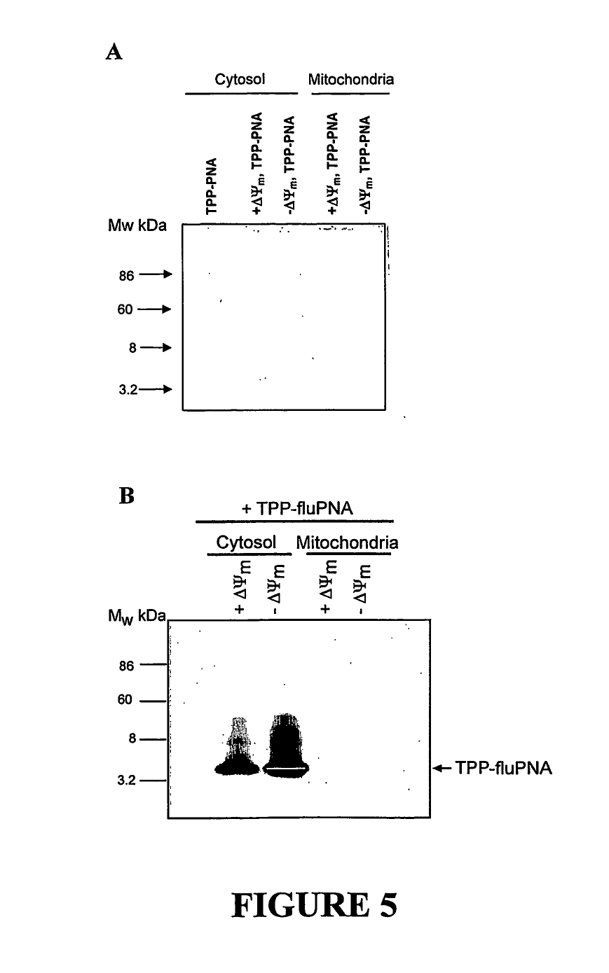

143B osteosarcoma cells (10⁶) were incubated with 1 μM TPP-PNA in the presence or absence of ΔΨₘ, (±10 μM FCCP) for 1 h at 37° C. and the cells were then separated by homogenisation with digitonin (1 mg.mL⁻¹) in mitochondrial and cytosolic fractions (FIG. 5A). These fractions were separated on non-reducing Tris-tricine gels, transferred onto nitrocellulose probed with streptavidin-linked horse radish peroxidase. PNA oligomers (5 nmol) were used as positive controls.

This procedure detected control PNA oligomer at the expected size (˜3.2 kDa). Cells incubated with the TPP-PNA conjugate accumulated the PNA oligomer in the cytoplasm. The absence of a mitochondrial membrane potential by treatment with the mitochondrial uncoupler FCCP did not affect the cytoplasmic distribution of the PNA oligomers. It was possible to confirm the presence of the PNA in the cell fractions by comparing them to a control PNA sample.

These experiments were repeated with P388 cells which confirmed the localisation of the PNA within the cytoplasm of cells independent of the mitochondrial membrane potential (FIG. 5 B).

The P388 cells were treated as described before, the lysates were resolved on Tris-tricine gels and the TPP-fluPNA conjugates were detected using a GelDoc fluorescence imager.

Cells treated with the TPP-PNA conjugate rapidly (within 30 min) accumulated large amounts of the PNA oligomer in the cytoplasm compared with unmodified PNAs (FIG. 5 C). The uptake of unmodified PNAs in cells most likely occurred by endocytosis as that has previously been shown to lead to the slow uptake of PNAs by cells. To test whether the phosphonium cation localized to mitochondria as a result of the large mitochondrial membrane potential, cytosolic fractions were probed using the anti-phosphonium serum (FIG. 5 D). TBTP was detected in the mitochondrial fractions where it reacted with thiol-containing proteins and labeled them with a phosphonium cation. The absence of TBTP-bound proteins in the cytosolic fraction suggests that the cation is rapidly accumulated in mitochondria (FIG. 5 D). These data indicate that TPP-PNA is taken up by cells, reduced in the cytoplasmic environment, leaving the antisense PNA free to bind its target mRNA.

### Confocal Immunofluorescent Microscopy of Fibroblasts

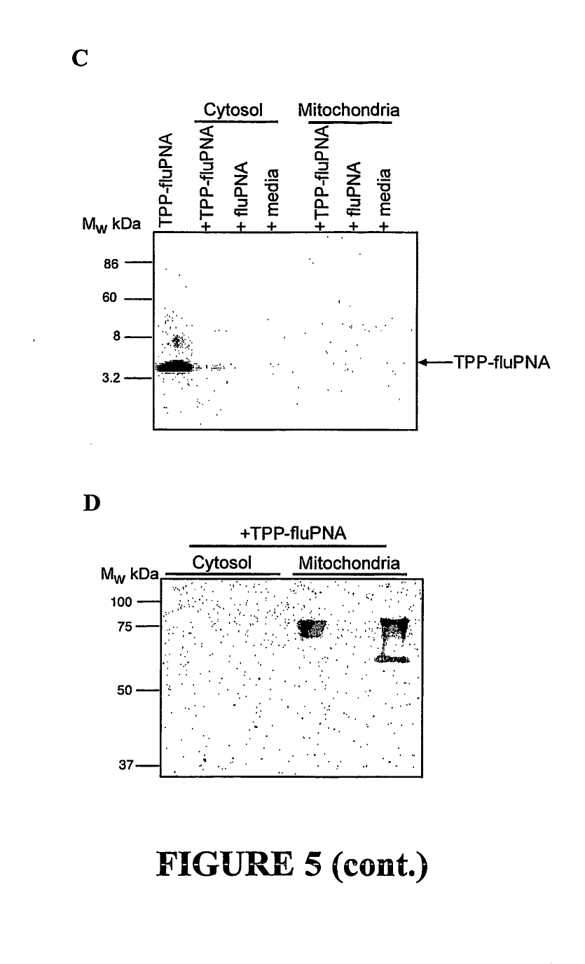

The localisation of TPP-PNA conjugate within human fibroblasts was also determined by fixing cells that had been incubated with TPP-PNA, PNA or without any additions for up to 4 h and visualising the localisation of the PNA by confocal immunofluorescent microscopy (FIG. 6). Cells were incubated with 1 μM TPP-bioPNA at 37° C. for 1 h (FIGS. 6A and 6C) and with 1 μM bioPNA for up to 4 h (FIGS. 6B and 6D).

Cells were fixed, incubated with antiserum against triphenylphosphonium (green) and a streptavidin-linked fluorophore to detect the biotin tagged PNA (red) and the images overlaid. (FIG. 6B). Overlay of these two micrographs showed that the PNA was distributed throughout the cytoplasm, while the phosphonium was confined to the mitochondria (FIG. 6A). This confirms that after delivery of the TPP-PNA to the cytoplasm it is reduced and the PNA remains in the cytoplasm, while the phosphonium cation is accumulated by the mitochondria. The unmodified PNA was also taken up by the cytoplasm and the nucleus but in far lower amounts (red) than the unmodified PNAs and only after a four hour incubation (FIG. 6B).

Cells treated with bioPNA for 30 min, 1 h and 4 h were fixed and incubated with antiserum against triphenylphosphonium, streptavidin-linked fluorophore (red) and the nucleus was stained with 4,6′-diamidino-2-phenylindole (DAPI, 3 μg.mL⁻¹) for 5 min (blue) to show the strict cytoplasmic localisation of the PNA (blue). Cells incubated with biotin PNA after showed uptake (red) only after 4 h. (FIG. 6C)

Live cells were incubated with TPP-fluPNAs for 1 h and the mitochondria were stained with MitoTracker (25 nM) for 15 min. (D) Cells treated with fluorescein PNA for 1 h and stained with MitoTracker. Magnification, 1400×. Scale bars, 20 μm.

Live cells treated with the TPP-fluPNA conjugates confirmed cytoplasmic localisation of the PNA. In addition, these experiments show that the cation effectively targeted at least 90% of the cells (FIG. 6C). Unmodified PNA accumulated very poorly into cells after an hour incubation (FIG. 6D).

These findings support the western blot data which also showed that low amounts of unmodified PNAs were taken up into the cytoplasm over a long incubation. In contrast the TPP-PNA delivery is both faster, and also delivers greater amounts of the PNA oligomers to the cytoplasm without trapping them in endosomes. Therefore the low ΔΨₚ is sufficient to drive accumulation of TPP-PNA in the cytoplasm where the disulfide bond is rapidly reduced, releasing the PNA. This system should be useful to deliver PNAs to the cytoplasm in order to inhibit the transcription or translation of genes of interest within cells.

### Down Regulation of PAX2 Protein Expression Using TPP-PNA Conjugates

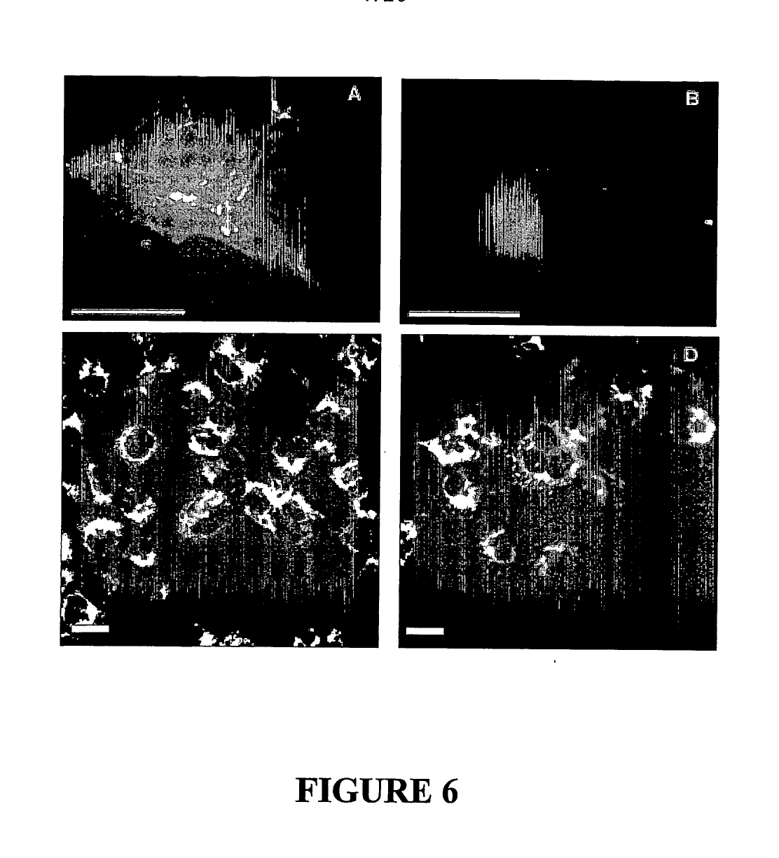

To investigate the biological effects of the anti-PAX2 TPP-fluPNA conjugate we used a mouse cell line that highly expresses the PAX2 protein. Treatment of P388 leukemia cancer cells with TPP-PNA conjugates resulted in a significant inhibition of PAX2 expression that lasted 4 days (FIG. 7). The knock down in PAX2 expression was specific and did not affect general cell proliferation (FIG. 7B). In contrast, treatment with unmodified anti-PAX2 PNA did not decrease PAX2 protein levels. This is most likely due to the absence of the cation that effectively delivers the PNA to the cells. Control experiments where cells were treated with media only showed the basal level of the PAX2 protein over 4 days.

In summary, the triphenylphosphonium cation facilitates efficient delivery of the neutral PNAs to the cytoplasm of cells where they can inhibit the translation of PAX2 mRNAs.

### EXAMPLE 2

PNAs targeting the mouse HNF4α mRNA were obtained from Applied Biosystems Inc. (Bedford, Mass.), and Santaris Pharma A/S (Denmark). The HNF4α PNA sequences were Flu-XX-GTCCCAGACGGT-Cys-COOH, where X is 8-amino-3,6-dioxanoic acid (Flu-PNA-TBTP, from Applied Biosystems Inc.) or Lys-GTCCCAGACGGT-Cys-COOH (Lys-PNA-TBTP, from Santaris Pharma A/S). The resulting conjugates were designated TPP-fluPNA and TPP-lysPNA.

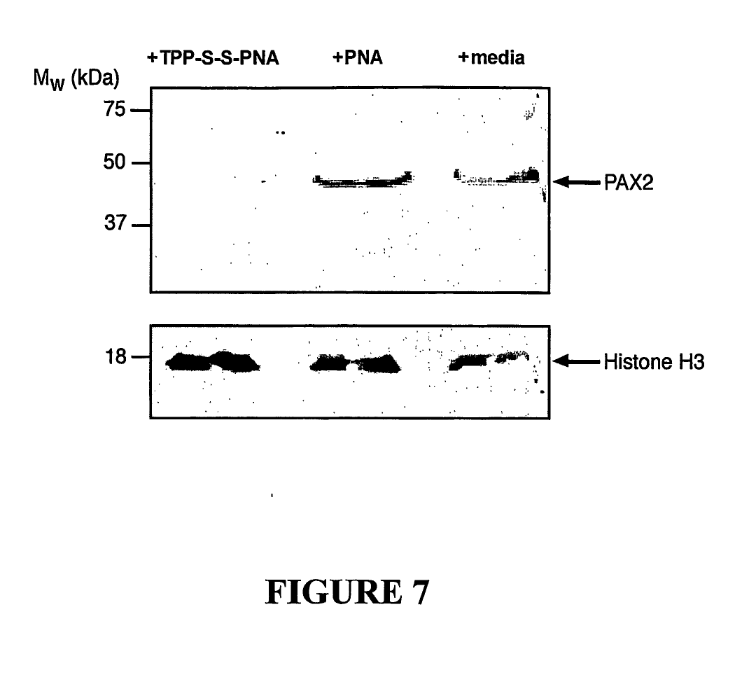

To synthesize the conjugates the PNAs were dissolved in 150 μL 10 mM HEPES, 1 mM EDTA, and were incubated with TCEP-HCl (250 nmol) at 37° C. for 30 min. To conjugate PNAs with TBTP, bisTBTP (250 nmol) in 20 μL 10 mM HEPES, 1 mM EDTA was added to the PNA solution and incubated at 37° C. for 1 h. Then H₂O₂ (440 μmol) in 50 μL of H₂O was added and the solution again incubated at 37° C. for 30 min. The reaction products were separated by RP-HPLC on a C4 analytical column (Phenomenex, 300A, 4.6 mm×150 mm), using a Waters 450 HPLC system and a linear gradient from 0.1% TFA, 2% acetonitrile in H₂O to 0.1% TFA, 80% acetonitrile was run over 60 min. The peaks were detected by absorbance at 260 nm, collected, and the peak eluting at 28 min was analyzed by MALDI-ToF mass spectrometry. The RP-HPLC profile of purification of TPP-fluPNA is shown in FIG. 8A and the MALDI-ToF mass spectrum in FIG. 8B.

### Cell Culture and Inverted Fluorescence Confocal Microscopy

A mouse liver cell line, BNL-CL2, was grown at 37° C. and 5% CO₂ in humidified atmosphere in Dulbecco's Modified Eagle's Medium supplemented with 10% inactivated FBS, 100 units.mL−1 penicillin, and 100 μg.mL−1 streptomycin, but without sodium pyruvate. To culture the cells with PNA, cells were seeded into a 96-well-plate, followed 16 h later by addition of 1 μM TPP-fluPNA alone or in an aqueous solution of 150 μM Chloroquine, and cultured for another 70 h. Images were acquired using a Zeiss inverted confocal microscope with 488 nm excitation wavelength light for tracking the TPP-fluPNA. Mitochondria specific dye MitoTracker Red CMXRos was added into cultured medium 2 h before taking confocal images using 578 nm excitation wavelength light.

### RT-PCR

Total RNA was extracted from cells cultured with either fluoresceinated or non-fluoresceinated TPP-PNA, or with PNA dissolved in 150 μM Chloroquine, or with medium controls for 24 h or 72 h. The concentration of extracted RNA was quantified using a NanoDrop spectrophotometer, and 1 μg of total RNA was reverse transcribed into cDNA using Invitrogen SuperScript III and random primers. The primers for PCR of mouse HNF4α were 5′-CAATGAATATGCCTGCCTCAA-3′ (forward primer) and 5′-ATTCAGATCCCGAGCCACTT-3′ (reverse primer).

### Results

### Purification and Characterization of TPP-PNA Conjugates

The TPP-PNA conjugates were purified by RP-HPLC. Purification of TPP-fluPNA is shown in FIG. 8A. The hydrophobic nature of the triphenylphosphonium moiety caused the conjugate to bind with higher affinity to the stationary phase, and to elute later than the unmodified PNA. The excess TBTP-SH eluted even later than the TPP-PNA conjugate, and bisTBTP eluted last. The peaks were detected at 260 nm and collected. The second large peak from the left (TPP-fluPNA) was analyzed by MALDI-TOF mass spectrometry and gave the predicted molecular mass (FIG. 8B)

### Uptake of Flu-PNA-TBTP By Cells

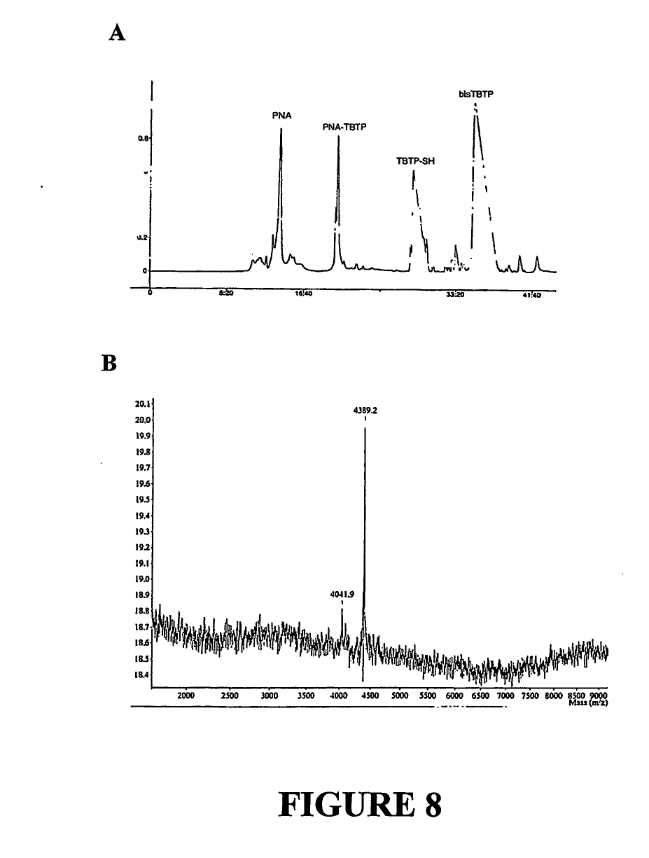

To observe the entrance of TPP-fluPNA into live cells, the BNL.CL2 cells were cultured with 1 μM of TPP-fluPNA for 4 h and images were recorded using inverted fluorescence confocal microscope. FIG. 9A showed that most of cells had TPP-fluPNA in the cytoplasm. To better identify whether this TPP-fluPNA was inside the mitochondria or other organelles, and whether PNA could enter into the nucleus, the PNA was incubated with cells for 44 h following which MitoTracker Red was added to the cultured cells 1 h before taking images. FIG. 9B showed that some cells had green fluorescence (fluorescein) in the nucleus, indicating that the PNA had translocated to the nucleus. As well as this, these cells had green fluorescence in the cytoplasm. Co-localization of mitochondria (red MitoTracker fluorescence) and PNA was not observed.

### Alternative Splicing of HNF4α mRNA

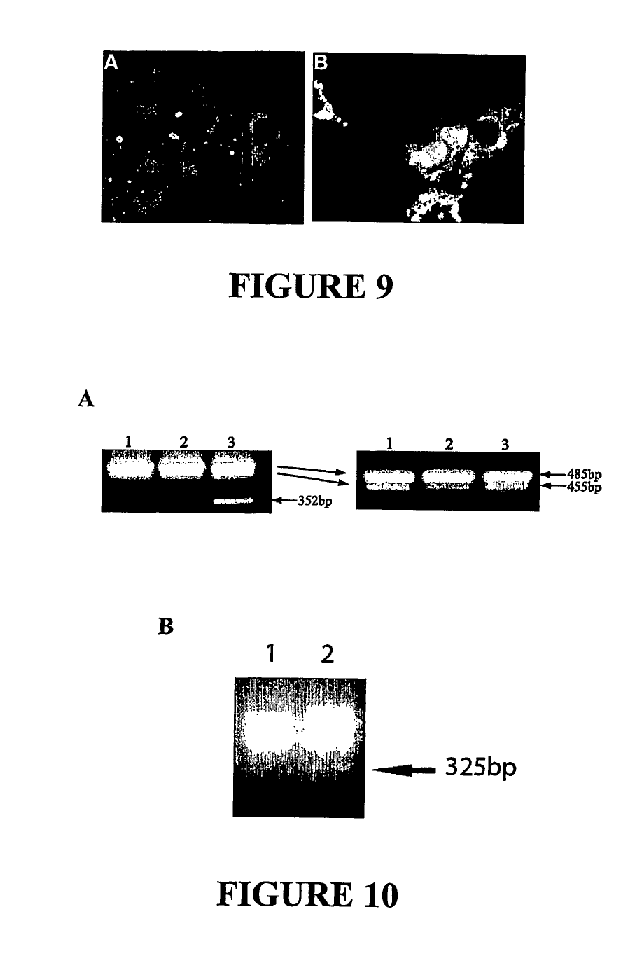

The HNF4α PNA was designed to target the intron 8/exon 9 junction of HNF4α pre-mRNA, and upon binding to the pre-mRNA it was predicted to invoke skipping of exon 9, giving rise to a splice variant in which exon 8 and exon 10 would be immediately adjacent to each other in the mature mRNA. The primers used in untreated BNL-CL2 cells gave two PCR fragments of 485 bp and 455 bp due to two naturally occurring splice variants at the 3′ end of exon 9. PCR amplification from a template derived from the exon 9-skipped mRNA was predicted to give rise to a product of 352 bp. This was confirmed by RT-PCR (FIG. 10A, lane 3). Chloroquine was able to induce uptake of PNA oligomer into BNL-CL2 liver cells, and so was used as a positive control. A 352 bp RT-PCR product was observed in addition to the 455 and 485 bp RT-PCR products in cells treated with HNF4α TPP-lysPNA and chloroquine, compared to the media control (FIG. 10, lanes 1 and 3). To detect whether extraneous PNA inside the cells could be co-purified in the extracted RNA and then inhibit the PCR reaction leading to the 352 bp RT-PCR product as an artifact, the same amount of PNA was added into cell lysis buffer before extraction of RNA, and the result was negative (FIG. 10A, lane2). Finally, to determine whether HNF4α TPP-lysPNA spontaneously entered liver cells unassisted by any transfection reagent, BNL-CL2 liver cells were treated with 1 μM HNF4α TPP-lysPNA in cell culture media for 70 h. A 352 bp RT-PCR product was observed in the treated cells, in addition to 455 and 485 bp RT-PCR products, compared to the media control (FIG. 10B). These data confirm that the HNF4α TPP-lysPNA enters the cells unassisted, translocates from the cytoplasm to the nucleus, and binds to HNF4α pre-mRNA, affecting splicing of this transcript.

### INDUSTRIAL APPLICABILITY

The strategy of targeting PNAs to the cell by conjugation to a triphenylphosphonium cation is an effective means of inhibiting gene expression. The cation facilitates transport of the PNA across the plasma membrane where it is taken up into the cytoplasm driven by the membrane potential. Once inside the cell, the disulfide bond is reduced in the cytoplasm, releasing the PNA oligomer. This prevents aggregation of the PNA in the mitochondria. The PNA may act in the cytoplasm to interfere with translation of mRNA or may make its way to the nucleus to interfere with transcription.

Intron-exon splicing is a crucial step in the processing of nearly all nuclear-encoded mRNA. PNAs have already been demonstrated to inhibit splicing of transcripts encoding particular proteins in cells, and so our results are in agreement with these studies. However, we have specifically shown here that PNA delivered to cells using the triphenylphosphonium cation is able to localize to the nucleus, and that it is bioactive in that it inhibits splicing of a specific exon thereby inducing exon skipping. The specificity of action of the released PNA in cells appears to be as high as an unmodified PNA. The outcome of exon skipping involving transcripts where an important exon has been deleted will often be frame-shifting of translation (the reading frame is disrupted), and therefore the transcript is rendered useless for the expression of the protein usually encoded by the gene. If engineered appropriately, new proteins could be produced, or the ratios of alternatively spliced proteins could be altered. Our results may also suggest that PNAs could be delivered to cells using the triphenylphosphonium cation to affect other types of pre-mRNA processing, or other RNA function, in the nucleus or elsewhere in the cell. For example, small nuclear RNA (SnRNA), ribo RNA (rRNA) and micro RNA (miRNA) perform many functions in the nucleus, cytoplasm and ribosomes, and these may also be targeted by PNAs.

In summary, the triphenylphosphonium cation facilitates efficient delivery of the PNAs into the cells, where the potentially cleavable disulfide bond tethering the PNA to the triphenylphosphonium cation is reduced, releasing the PNA to become available and active within non-mitochondrial sites and organelles. Not only can neutral PNAs be delivered to cells, but also PNAs bearing at least one amino acid such as lysine carrying a positive charge. The data shown demonstrate that PNAs are available to block translation of mRNA at ribosomes in the cytoplasm (eg inhibition of PAX2 translation), and that they can alter RNA processing in the nucleus (eg exon-skipping and therefore altered splicing during HNF4α transcription).

This conjugate delivery system of the present invention has some advantages over conventional delivery methods: (i) the uptake is rapid and occurs directly through the membrane without the need for receptor-mediated uptake or endo- or pinocytosis and (ii) the phosphonium uptake is indiscriminant of cell type, and (iii) the conjugate is not cytotoxic in micromolar concentrations and is not degraded within the cell.

Delivery of PNAs to the cytoplasm by conjugation to disulphide linked lipophilic cations also has the advantage of being simple, cheap and effective.

Accordingly, the conjugates of the invention have potential in medical applications such as the treatment of bacterial and viral infections, cancer, metabolic diseases, immunological disorders and the like.

Triphenylphosphonium-linked delivery of PNAs may also be used as a research tool. For example, cell array technology (Wu, 2000), could be used to develop libraries of PNAs that could be delivered to cells for the purposes of drug discovery. Cell culture plates/slides containing spotted/arrayed individual TPP-PNAs (with thousands or hundreds of thousands of different sequences) could serve as a base for cells to be plated in such a way as to cover the arrayed TPP-PNAs like a blanket. The cells could contain a reporter of some sort, e.g., a gene promoter-reporter construct so as to assay for specific PNA sequences that disrupt a particular transcription factor's interaction with its target. The TPP-PNA would enter the cells located directly over the arrayed spot but wouldn't enter any other cells on the slide. The result of the uptake of the TPP-PNA into cells would be measured by the reporter inside the cells and detected in the array (on a spot-by-spot basis), using a scanning detector able to detect the signal from the reporter in the cells.

This approach is not limited to analysis of effects of PNAs on gene activity. As the reporter could be anything giving a detectable signal, and need not be genetic in nature, the PNAs could influence reporter activity by non-genetic mechanisms, for example, by disrupting protein-protein interactions in the cytoplasm.

The TPP-PNA conjugates may be found to disrupt or modulate many kinds of cellular process through interaction with gene transcription mechanisms. For example, certain PNAs may be able to bind to DNA and prevent certain proteins from defining MARs (matrix attachment regions) during the early commitment of stem cells into cell lineages. This may have the effect of re-designing the chromatin packaging of a cell, and as a result the subsequent gene activity, thereby modulating how much, or what type of cells are determined in a tissue. This is possibly the mechanism by which some teratogenic agents act to cause birth defects.

The conjugates of the invention may also be used in imaging techniques, for example, in techniques that allow the imaging of oncogene expression. For example, radionuclide-PNA conjugates can be used to detect over-expression of mRNAs that are markers of oncognic transformation (Tian et al. 2003).

It is to be understood that the scope of the invention is not limited to the examples described above and therefore that numerous variations and modifications may be made to the described embodiments without departing from the scope of the invention.

### REFERENCES

1. Egholm, M., Buchardt, O., Christensen, L., Behrens, C., Freier, S. M., Driver, D. A., Berg, R. H., Kim, S. K., Norden, B., and Nielsen, P. E. (1993) Nature 365, 566-568
2. Demidov, V. V., Potaman, V. N., Frank, K. M., Egholm, M., Buchard, O., Sonnichsen, S. H., and Nielsen, P. E. (1994) Biochemical Pharmacology 48, 1310-1313
3. Simmons, C. G., Pitts, A. E., Mayfield, L. D., Shay, J. W., and Corey, D. R. (1997) Bioorganic and Medicinal Chemistry Letters 7, 3001-3006
4. Eriksson, M., Nielsen, P. E., and Good, L. (2001) J Biol Chem 5, 5
5. Muratovska, A., Lightowlers, R. N., Taylor, R. W., Turnbull, D. M., Smith, R. A. J., Wilce, J. A., Martin, S. W., and Murphy, M. P. (2001) Nucleic Acids Res 29(9), 1852-1863.
6. Burns, R. J., Smith, R. A. J., and Murphy, M. P. (1995) Arch Biochem Biophys 322(1), 60-8.
7. Ellman, G. and Lysko, H. (1979) Anal Biochem 93, 98-102
8. Berry, M. N., Edwards, A. M., Barritt, G. J., Grivell, M. B., Halls, H. J., Gannon, B. J., and Friend, D. S. (1991) Isolated hepatocytes preparation, properties and applications, Amsterdam: Elsevier
9. Schagger, H., and von Jagow, G. (1987) Analytical Biochemistry 166, 368-379
10. Ljungstrom, T., Knudsen, H. and Nielson, P. E. (1999) Bioconjugate Chemistry 10, 965-972.
11. Wu, R. Z. (2002) Trends in Cell Biology 12, 485-488.
12. Eriksson, M. and Nielsen, P. E. (1996) Q. Rev. Biophys. 29(4) 369-394.
13. Knudsen, H. and Nielson, P. E. (1997) Anticancer Drugs 8(2) 113-118.
14. D'Costa, M.; Kumar, V. A.; Ganesh, K. N. Organic Lett. 1999, 1 (10), 1513-1516.
15. Mischiati, C.; Borgatti, M.; Bianchi, N.; Rutigliano, C.; Tomassetti, M.; Feriotto, G.; Gambari, R. J. Biol. Chem. 1999, 274 (46) 33114-33122.
16. Borgatti, M.; Breda, L.; Cortesi, R.; Nastruzzi, C.; Romanelli, A.; Saviano, M.; Bianchi, N.; Mischiati, C.; Pedone, C.; Gambari, R. Biochemical Pharmacology 2002, 64, 609-616.
17. Tian, X.; Aruva, M. R.; Rao, P. S.; Qin, W.; Read, P.; Sauter, E. R.; Thakur, M.

L.; Wickstrom, E. Ann. N.Y. Acad. Sci. 2003, 1002: 165-188.

All references cited throughout the specification and this reference listing are herein incorporated by reference.

1-30. (canceled)

31. A conjugate of formula I 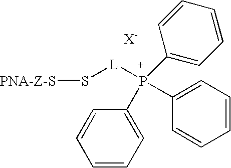
wherein L is a linker group, S-Z is a thiol-containing attachment group, X⁻ is an optional anion, and PNA is a peptide nucleic acid.

32. The conjugate according to claim 31 wherein L is (C₁-C₃₀) alkylene or substituted (C₁-C₃₀) alkylene.

33. The conjugate according to claim 32 wherein L is (C₃-C₁₀) alkylene.

34. The conjugate according to claim 33 wherein L is butylene.

35. The conjugate according to claim 31 wherein Z is selected so that S-Z is a cysteinyl, homocysteinyl or an aminothiol compound attached to a suitable linking group for linking to the PNA residue.

36. The conjugate according to claim 31 wherein the linking group for linking to the PNA residue is 8-amino-3,6-dioxanoic acid.

37. The conjugate according to claim 31 wherein PNA is a PNA oligomer targeting either a unique region in both the mouse and human PAX2 mRNA or mouse HNF4α.

38. The conjugate according to claim 37 wherein PNA is TTCACACCCCCGTGCC, GTCCCAGACGGT or lys-GTCCCAGACGGT.

39. The conjugate according to claim 31 wherein the PNA is attached to a molecular tag or reporter molecule.

40. The conjugate according to claim 39 wherein the molecular tag or reporter molecule is an affinity label.

41. The conjugate according to claim 40 wherein the affinity label is streptavidin or biotin.

42. The conjugate according to claim 39 wherein the reporter molecule is fluorescein.

43. The method of synthesizing a TPP-PNA conjugate according to Formula I, as defined in claim 31, comprising:
(a) incubating a compound of Formula II, wherein L and X are defined as above, 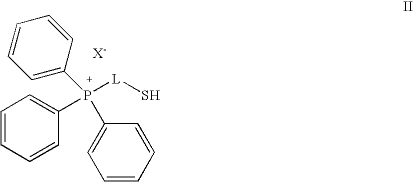
with an oxidant, to form the disulphide compound of Formula III 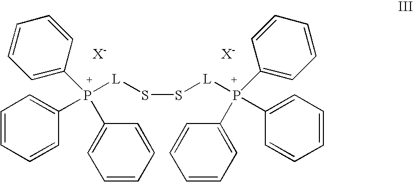
(b) reacting the compound of Formula III from step (a) with a compound of Formula IV
PNA-Z-SH IV
wherein Z and PNA are defined as above, and wherein the compound of Formula IV has been preincubated with a non-thiol containing reducing agent, to form the TPP-PNA conjugate of Formula I.

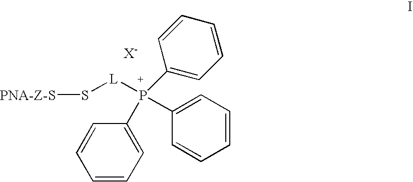

44. The method according to claim 43 wherein L is (C₁-C₃₀) alkylene or substituted (C₁-C₃₀) alkylene.

45. The method according to claim 44 wherein L is (C₃-C₁₀) alkylene.

46. The method according to claim 45 wherein L is butylene.

47. The method according to claim 43 wherein Z is selected so that S-Z is a cysteinyl, homocysteinyl or an aminothiol compound attached to a suitable linking group for linking to the PNA residue.

48. The method according to claim 43 wherein the linking group for linking to the PNA residue is 8-amino-3,6-dioxanoic acid.

49. The method according to claim 43 wherein PNA is a PNA oligomer targeting either a unique region in both the mouse and human PAX2 mRNA or mouse HNF4α.

50. The method according to claim 49 wherein PNA is TTCACACCCCCGTGCC, GTCCCAGACGGT or lys-GTCCCAGACGGT.

51. The pharmaceutical composition comprising a therapeutically effective amount of a compound of Formula I, as defined in claim 31, in combination with one or more pharmaceutically acceptable excipients, carriers or diluents.

52. The method of treating a patient with a disease or disorder that is susceptible to antisense therapy, which comprises the step of administering to said patient, a therapeutically effective amount of a compound of Formula I, as defined in claim 31.

53. The method according to claim 52 wherein the disease or disorder is selected from the group comprising bacterial infections, viral infections, cancer, metabolic diseases and immunological disorders.

54. The method according to claim 52 wherein the disease or disorder is selected from the group comprising HIV infection, hepatitis C infection; melanoma, pancreatic adnocarcinoma, actue myeloid leukemia, myeloma, small cell lung cancer, prostate cancer, ovarian carcinoma, breast cancer, glioma; hypercholesterolemia and amyloid light chain amyloidosis.

55. The method of targeting PNA oligomers to non-mitochondrial sites or organelles within a cell, including the cytoplasm and/or the nucleus, using a compound of Formula I as defined in claim 31, said method comprising delivering the PNA oligomers across the plasma membrane, without promoting selective aggregation in the mitochondria of said cell.

56. The method for modifying gene expression by administering a compound of Formula I as defined in claim 31, to a cell.
